(19) United States
(12) Patent Application Publication
(10) Pub. No.: US 2024/0318225 A1
(43) Pub. Date: Sep. 26, 2024

(54) TECHNIQUES FOR ISOLATION OR ANALYSIS OF BACTERIAL PATHOGENS FROM PATIENT SAMPLES

(71) Applicants: General Electric Company, Schenectady, NY (US); The Penn State Research Foundation, University Park, PA (US)

(72) Inventors: Christopher Michael Puleo, Niskayuna, NY (US); Erik Leeming Kvam, Niskayuna, NY (US); Pak Kin Wong, State College, PA (US); Peter Torab, Edinboro, PA (US); Christine Lynne Surrette, Albany, NY (US)

(21) Appl. No.: 18/293,305

(22) PCT Filed: Jul. 29, 2022
(86) PCT No.: PCT/US2022/038792

§ 371 Date: Jan. 29, 2024

Related U.S. Application Data
(60) Provisional application No. 63/227,717, filed on Jul. 30, 2021.

(51) Int. Cl. C12Q 1/24; C12Q 1/18; G01N 33/49

(52) U.S. Cl. CPC C12Q 1/24; C12Q 1/18; G01N 33/491

(57) ABSTRACT

The subject matter of the present disclosure generally relates to techniques for isolating bacterial cells from a biological sample comprising red blood cells. Using an aggregating agent and an anticoagulant during sedimentation permits separation of bacterial pathogens in the sample from red blood cells. The separated sedimentation layer, which is enriched in any bacterial pathogens, can be centrifuged and resuspended to concentrate the bacteria for additional analysis, such as bacterial identification and/or antibiotic susceptibility tests.

### STATEMENT OF GOVERNMENT SUPPORT

This invention was made with U.S. Government support under Agreement No. W15QKN-16-9-1002 awarded by the United States Army/ACC-NJ. The Government has certain rights in the invention.

### BACKGROUND

The subject matter disclosed herein relates to techniques for isolation and enrichment of pathogens from whole blood. In particular, the disclosed techniques relate to methods, systems, and compositions that improve pathogen recovery from sedimentation workflows and that can be used in conjunction with subsequent identification and/or susceptibility analysis.

Bloodstream infections, which may lead to sepsis, shock, and other life-threatening complications, are major global healthcare challenges. Timely identification of bloodborne pathogens is a recognized clinical bottleneck in the management of these infections. For example, vials of blood are drawn from patients and cultured for up to five days to detect the presence of pathogens. If the culture is positive, samples from the cultures are used for Gram staining and molecular analysis (e.g., polymerase chain reaction) of pathogens to identify the species. Thus, culture and identification of pathogens present in patient samples may take several days. The confirmation of a bacterial infection and identification of the bacterial species can facilitate the selection of a pathogen-specific treatment based on antimicrobial susceptibility testing. Due to these time-consuming processes to obtain both microbial identification and antimicrobial susceptibility testing from blood, patients are often prescribed broad spectrum antibiotics prior to obtaining a precise diagnosis. However, precise antibiotic treatments, in contrast to broad spectrum antibiotics, are more effective and can minimize the disruption of the commensal microbiota, which improve the clinical outcome. Unfortunately, the prolonged delay in microbiological diagnosis promotes non-targeted usage of antibiotics, which may result in less effective patient treatment and may also facilitate the emergence of antibiotic-resistant pathogens.

### BRIEF DESCRIPTION

Certain embodiments commensurate in scope with the originally claimed subject matter are summarized below. These embodiments are not intended to limit the scope of the claimed subject matter, but rather these embodiments are intended only to provide a brief summary of possible embodiments. Indeed, the invention may encompass a variety of forms that may be similar to or different from the embodiments set forth below.

In one embodiment, a method to isolate bacterial cells from a biological sample comprising red blood cells is provided. The method includes the steps of contacting the biological sample with an aggregating agent and an anticoagulant to form a sedimentation solution having a first volume in a sample processing container; allowing gravimetric sedimentation to occur such that a top layer and a bottom layer are formed in the sample processing container, wherein the bottom layer is enriched in red blood cells relative to the top layer; isolating the top layer from the bottom layer; centrifuging the isolated top layer to form a pellet; separating the pellet from a supernatant; and resuspending the separated pellet in a second volume of suspension solution to form a sample solution, the second volume being smaller than the first volume.

In one embodiment, a kit to sediment cells in a biological sample is provided. The kit includes an aggregating agent, the aggregating agent have a molecular weight of at least 100 kDa; and an anticoagulant.

In one embodiment, a method to analyze whole blood is provided. The method includes the steps of forming a top layer and a bottom layer via gravimetric sedimentation in a sample processing container comprising a biological sample, an aggregating agent and an anticoagulant, wherein the bottom layer is enriched in red blood cells relative to the top layer; centrifuging the top layer to form a pellet; resuspending the separated pellet in a second volume of suspension solution to form a sample solution; and analyzing the sample solution for a presence of bacteria.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1A is a schematic illustration of the sample preparation workflow with dextran sedimentation and centrifugation for single cell antimicrobial susceptibility testing of bloodstream infection according to embodiments of the disclosure;

FIG. 1B is a schematic illustration of a microfluidic device for single cell antimicrobial susceptibility testing and a (bottom) zoom-in view of bacteria trapped in the microchannel according to embodiments of the disclosure;

FIG. 1C shows bacteria trapped in the microscale channel of a single cell microfluidic device for visualizing the presence of bacteria and their response to antibiotics and showing a scale bar of 20 μm;

FIG. 1D shows (top and bottom) images of individual bacteria trapped in the microfluidic channel. and showing scale bars of 5 μm;

FIG. 2A is a schematic illustration of the dextran sedimentation protocol for removal of blood cells according to embodiments of the disclosure;

FIG. 2B shows results for blood cell removal efficiency showing that the cell count decreases exponentially with the sedimentation time in the plasma and an inset showing the data plotted in semi-log scale to illustrate cell removal for several orders of magnitude;

FIG. 2C shows bacteria recovery (portion of total bacteria) with 15 minutes of dextran sedimentation;

FIG. 2D shows bacteria recovery (portion of total bacteria) with 30 minutes of dextran sedimentation;

FIG. 2E shows bacteria recovery and sample volume reduction by a soft spin at 200 g for 20 min;

FIG. 3A shows recovery rates of E. coli, E. faecalis, K. pneumoniae, and S. aureus after dextran sedimentation;

FIG. 3B shows control experiments with plasma and dextran solution for characterizing the recovery rate of S. aureus;

FIG. 3C shows recovery rates of S. aureus with a thrombin inhibitor, argatroban after 30 min dextran incubation;

FIG. 3D shows recovery rates of E. coli with a thrombin inhibitor, argatroban after 30 min dextran incubation;

FIG. 4A shows phenotypic growth of K. pneumoniae in buffer, 10% blood, and dextran separated plasma;

FIG. 4B shows phenotypic growth of E. faecium in buffer, 10% blood, and dextran separated plasma;

FIG. 5 shows growth of isolated E. coli in single cell microchannels with varying concentrations of ampicillin;

FIG. 6 is a flow diagram of a method of isolating bacteria from a biological sample having red blood cells according to embodiments of the disclosure; and

FIG. 7 is a schematic illustration of a kit to sediment cells in a biological sample according to embodiments of the disclosure.

### DETAILED DESCRIPTION

Bloodstream infections are a significant cause of morbidity and mortality worldwide. Rapid initiation of effective antibiotic treatment is critical for patients with bloodstream infections. However, the diagnosis of blood-borne pathogens is largely complicated by the matrix effect of blood and the lengthy blood tube culture procedure. Due to the low bacteria load, single cell analysis is particularly attractive for diagnosis of bloodstream infections without the blood tube culture step. Nevertheless, bloodstream infection diagnosis remains challenging due to the low bacteria concentration and the complex matrix effect of blood. Sample preparation procedures based on centrifugation and filtering have been developed to isolate bacteria from whole blood. However, the difficult manual steps associated with these techniques and pathogen species specific challenges (such as filter interactions or pathogen-host cell interactions) often make clinical translation of these technique impractical.

In particular, single cell analysis platforms are highly promising for providing high resolution diagnosis with a quick turnaround time. For example, automated single cell morphological analysis platforms with machine learning algorithms provide cost-effective and accurate antimicrobial susceptibility data in non-traditional healthcare settings. A nanoarray digital polymerase chain reaction with high resolution melt curve analysis enables rapid broad bacteria identification and phenotypic antimicrobial susceptibility testing. Furthermore, single cell microfluidic devices along with molecular biosensors allow rapid classification of the pathogen, detection of polymicrobial samples, identification of bacterial species, and single cell antimicrobial susceptibility testing. These platforms have been demonstrated for rapid diagnosis of various common infection, such as urinary tract infections and wound infections. Effective sample preparation procedures that bypass the lengthy blood culture step are, therefore, highly sought-after for single cell microbiological analysis of bloodstream infections.

Provided herein are techniques for rapid isolation and enrichment of pathogens, such as bacterial or other microbial pathogens (e.g., fungi, parasites) from whole blood. The techniques can be used in conjunction with single cell microbiological analysis. In an embodiment, a dextran sedimentation step is used to reduce the concentration of blood cells from a whole blood sample. In an embodiment, the incorporation of matrix disrupting agents, such as anti-coagulants, can further improve sedimentation. Red blood cell depletion in the recovered bacteria after sedimentation facilitates the downstream centrifugation-based enrichment step at a sepsis-relevant bacteria concentration. The disclosed techniques are compatible with common antibiotic-resistant bacteria and do not influence the minimum inhibitory concentrations used in susceptibility testing, e.g., rapid single cell testing using microfluidic devices.

The disclosed techniques can be used in conjunction with a culture-free workflow for bloodstream infection diagnostics, such as a workflow for isolating common antibiotic-resistant bacteria from whole blood. The workflow involves relatively simple equipment and procedures, which can be potentially implemented in non-traditional settings, such as in the field. If the resources (e.g., power) are limited, portable and hand-powered centrifuges can be considered to simplify the system requirement further, as the sedimentation step requires only a single volume reduction centrifugation step. Further, the techniques permit effective sedimentation using 1) a single volume reduction sedimentation step that is 2) at relatively lower speeds, such that lysis of recovered bacteria cells is reduced relative to workflows that use higher spin speeds and/or multiple centrifugation steps. The isolation and enrichment steps may be finished in approximately 30 minutes, which is similar or faster than other diagnostic workflows. Using a microfluidic device capable of single cell analysis, such as a microfluidic device as disclosed in WO 2020/014537, which is incorporated by reference herein for all purposes, pathogen classification can be performed in as fast as 5 minutes by microscopic examination, and antibiotic susceptibility results can be obtained in a timescale similar to the doubling time of the pathogen. Use of a microfluidic device also standardizes the broth volume, which minimizes the influence of the inoculum effect, and promotes rapid bacteria growth by facilitating gas exchange. Importantly, the workflow maintains the viability of the bacteria and is compatible with other single cell microbiological analysis platforms, including machine learning-based morphological analyzers and microfluidic molecular assays.

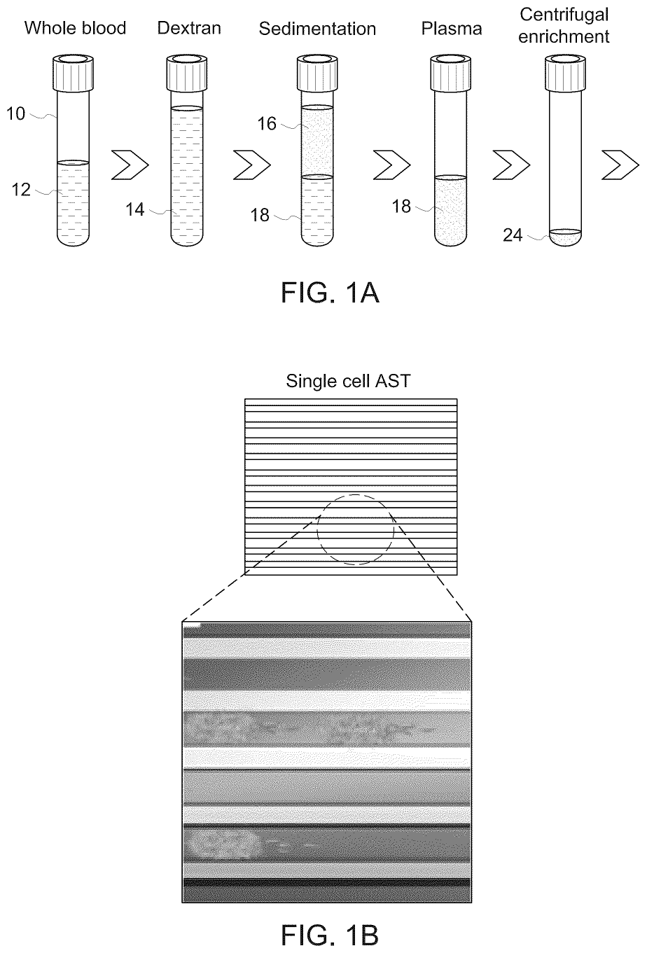

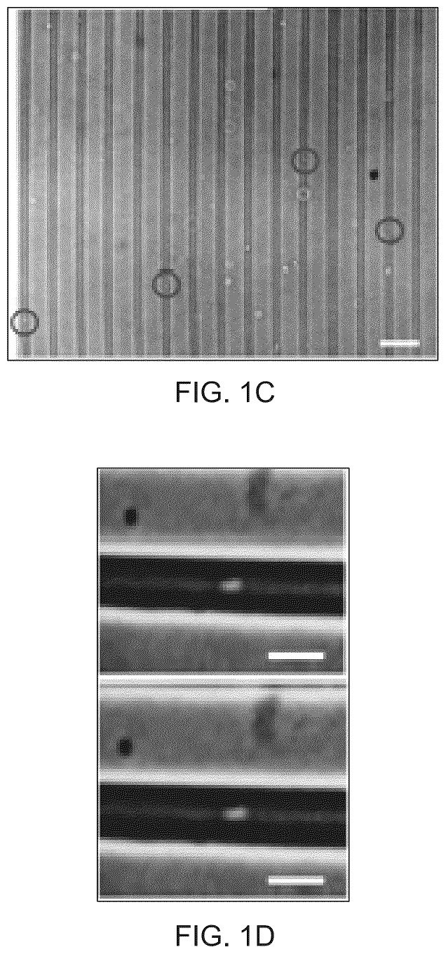

FIG. 1A is a schematic illustration of the sample preparation workflow with dextran sedimentation and centrifugation for single cell antimicrobial susceptibility testing of bloodstream infection. The workflow of FIG. 1A was used to obtain results of the examples disclosed herein. The workflow starts with dextran sedimentation for red blood cell (erythrocyte) depletion, which has been applied for immunological analysis, such as neutrophil purification, and other biomedical applications. The dextran-isolated bacteria are then enriched by centrifugation, providing an effective method for red blood cell depletion compared to the complex selective lysis or gradient centrifugation techniques currently employed. The enriched sample is loaded into a microfluidic device for determining the presence of bacteria in the sample and phenotypic single cell antimicrobial susceptibility testing.

More specifically, in the workflow shown in FIG. 1A, a biological sample 12 that includes red blood cells is provided in a container 10. A dextran solution and sodium polyanethole sulfonate are mixed with whole blood to form a sedimentation solution 14. The mixture, or solution 14, is then allowed to settle and sediment for, in an embodiment, less than 30 minutes to deplete the red blood cells. Two layers are formed, a top layer 16 (e.g., a supernatant phase) and a bottom layer 18 (e.g., a sedimented phase). The top layer 16 is depleted of red blood cells, while the bottom layer 18 includes more blood cells. The plasma (top layer 16 or clear portion of the solution) is then pipetted out carefully to recover portion with bacterial pathogens. Second, the plasma is further enriched by centrifugation. The enrichment step produces a sample 24 with reduced sample volume and increases the concentration of bacteria for microfluidic single cell analysis.

With a majority of red blood cells removed by the simple sedimentation step into the bottom layer 18, centrifugation becomes a one-step process to achieve volume reduction, instead of the multi-step process required if using common selective lysis or gradient centrifugation methods for bacteria selection. After removal of the supernatant, is pellet resuspended, e.g., in 50 microliters of MH broth. In the disclosed results, all reagents were obtained from Sigma (St. Louis, MO) unless otherwise noted. Pathogenic bacteria isolates (Escherichia coli, Klebsiella pneumoniae, Enterococcus faecails, and Staphylococcus aureus) were isolated from patient urine samples under an approved protocol from the Stanford University Institutional Review Board. The antimicrobial resistance profiles for pathogenic E. coli were previously determined by the clinical microbiology laboratory at Veterans Affairs Palo Alto Health Care System. E. faecium was obtained from ATCC (ATCC 35667).

To isolate and enrich bacteria from whole blood, the dextran and sodium polyanethole sulfonate (SPS) solution were first filtered using a PES membrane with 0.2 μm pore size. The bacterial sample was diluted to 2×10⁵ cfu/mL, and the appropriate volume was spiked into the blood solution to control the concentration (10-100 cfu/ml). The mixture included 10 mL of whole blood, 12 mL of 2.25% 500 kDa dextran solution (Spectrum D1004), and 1.98 mL of 1% SPS solution. The mixture was allowed to sediment at room temperature until a clear plasma-like layer (referred to as plasma layer below) was formed (˜15-30 min). This top plasma layer was removed and mixed with a pipette to ensure equal distribution of bacteria. The plasma layer was separated into 4 tubes, each containing a volume ˜1 mL. Each tube was centrifuged at 2000 g for 5 min (Denville 260D Brushless Centrifuge). The upper layer, or supernatant, was removed, and the pellet containing bacteria and any human cells not removed in the sedimentation step was resuspended in 0.1 mL of Mueller-Hinton (MH) broth. Bacteria counts were determined by plate counting, and recovery rates were estimated by the portion of recovered bacteria relative to the amount of bacteria spiked into the samples.

In the disclosed embodiments, the sample 24 the sample was loaded into microchannels with cross-sectional dimension compatible to the characteristic length (e.g., width) of the bacteria as shown in FIG. 1B. The channel functioned as a filter to separate the sample matrix (cell debris or other cell components) and enabled visualization and enumeration of bacteria in the sample, as shown in FIGS. 1C and 1D. The microfluidic channel also trapped the bacteria to facilitate monitoring of the bacteria response to antibiotics (i.e., phenotypic antimicrobial susceptibility testing). The blood-to-antimicrobial susceptibility testing workflow may be completed in less than 2 hours compared to 5-7 days using conventional blood tube culture-based techniques in clinical laboratories. Thus, the disclosed techniques permit faster and effective analysis of bacterial pathogens in whole blood samples.

As disclosed herein, a microfluidic device was incorporated for analyzing bacteria in the separated plasma. The microchannel assists visualization of individual bacteria, determines the presence of bacteria, and performs antimicrobial susceptibility testing phenotypically. However, a challenge of direct blood analysis is the low bacteria concentration (10⁰-10¹ cfu/mL). Since the microfluidic antimicrobial susceptibility testing device handles only 5-50 μL of fluid, the effective bacteria count could be less than 1 cfu. Therefore, a centrifugation step was incorporated to enrich the sample through volume reduction. The recovery rate of the centrifugation step was determined to be over 80% based on the plate count method. Enriched samples were then directly loaded into the inlet of the microfluidic devices for bacterial trapping. Since the microchannel height (1.3 μm) was compatible with the size of a bacterium, bigger objects, e.g., blood cells, were effectively filtered out by the channel. Without the dextran sedimentation step, filtering by the microchannel, however, was not possible due to clogging of the channel by the blood cells. The presence of viable bacteria in the sample was determined by microscope inspection of the motility and growth of the bacteria. As shown in FIG. 1C and FIG. 1D, trapping of bacteria was demonstrated in blood samples with as low as 10 cfu/ml. Since blood is generally sterile, the presence of bacteria can provide a direct indication of bacterial infection. The use of multiple channel heights within the microchannel device can be used for size-based classification of the bacteria in conjunction with the disclosed techniques.

The microfluidic device for single cell antimicrobial susceptibility testing was fabricated by soft lithography. The microchannel master mold was fabricated by photolithography patterning and reactive-ion etching of a silicon wafer. Microchannel layers were then fabricated by PDMS molding on the master mold. PDMS pre-polymer and cross-linker were mixed at 10:1 ratio. The mixture was poured on the master mold and incubated for at least 3 hours at 65° C. The single cell antimicrobial susceptibility testing device was fabricated by bonding the PDMS layer with a glass slide. Inlet and outlet reservoirs were created by punching the PDMS layer with a biopsy puncher.

To perform the microfluidic single cell antimicrobial susceptibility testing experiment, ampicillin was added to the enriched samples with concentrations of 0 μg/mL, 2 μg/mL, 4 μg/mL, and 8 μg/mL. Each respective solution was loaded into a microfluidic device by capillary force. The devices were then mounted onto an epi-fluorescence microscope (Leica DMI 4000B, objective 20× or 40×) with a microscope heating stage. The presence of bacteria was examined, and the bacterial growth was monitored continuously.

Data analyses were performed with Excel. The data were analyzed using one-way analysis of variance and Tukey's post-hoc test. Data represent mean±s.e.m. A two-sided p-value of <0.05 was considered statistically significant.

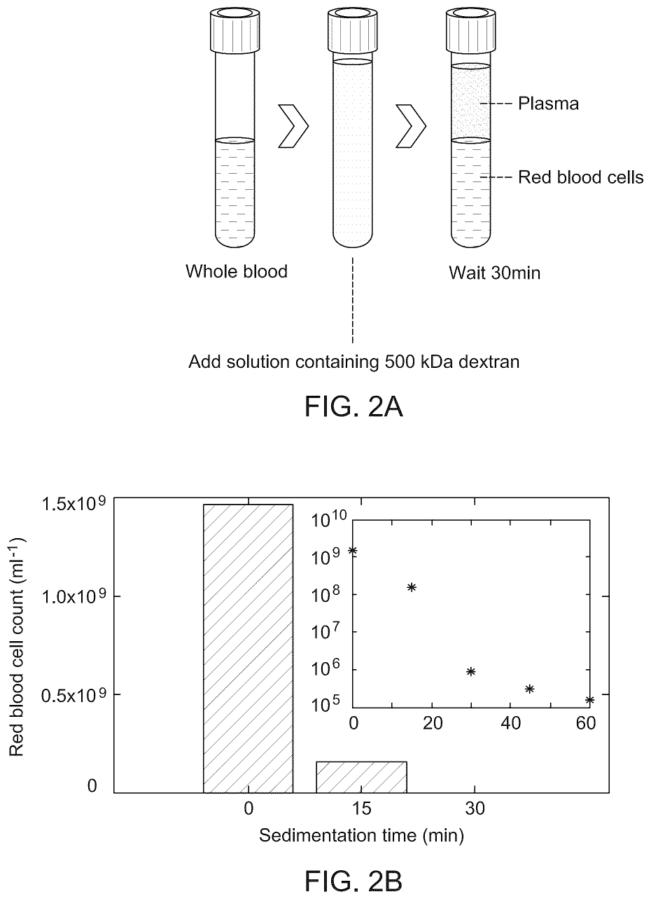

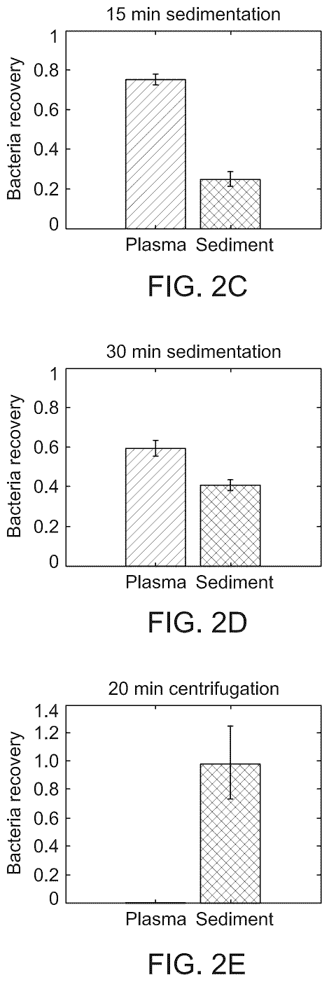

Red blood cell depletion efficiency of the dextran sedimentation step of FIG. 1A and using the dextran sedimentation protocol for removal of blood cells pf FIG. 2A was evaluated using E. coli, and the results are shown in FIG. 2B-E. The concentration of red blood cells was measured as a function of the sedimentation time by cell counting with a hemacytometer. As shown in FIG. 2B, the initial concentration of red blood cells was on the order of 10⁹ cells per ml. The red cell counts dropped to 10⁵-10⁶ cells per ml in the first 30 minutes. After that, the blood cell count further reduced at a slower rate (FIG. 2B inset). In contrast, the clear portions of the solution after 15 to 30 minutes of sedimentation retained the majority (>50%) of the amount of the spiked bacteria (FIG. 2C-D). As a result, sedimentation times of less than 30 minutes are associated with retention of most of bacteria in a biological sample in a plasma, or top layer 16. Recovery of about 50% of bacteria of plasma by volume, the effective starting concentration of the bacteria in blood (per ml) was similar to the initial concentration while the majority of red blood cells was removed. This is a substantial enhancement compared to direct centrifugation of the bacteria. The specific gravities of human red blood cells and E. coli are both around 1.1 g/ml. Due to their similarity in specific gravity, direct centrifugation resulted in a considerable loss of bacteria into the sediment portion (FIG. 2E). In contrast, the dextran sedimentation step allowed depletion of red blood cells while maintaining the effective concentration of bacteria in the sample. The data represent mean±s.e.m (n=3).

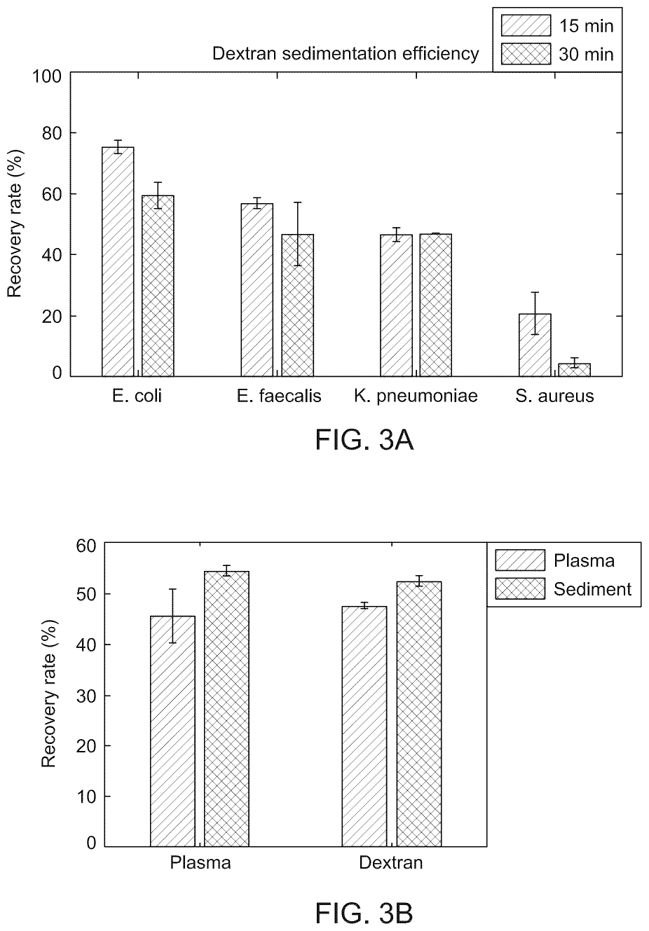

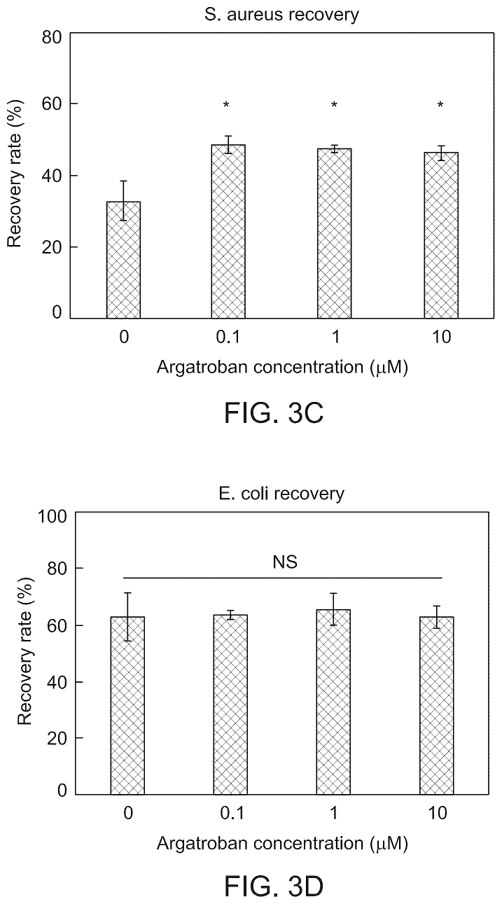

To evaluate the applicability of the dextran sedimentation step for bloodstream infection diagnostics, the procedure was performed in human whole blood samples spiked with several clinical bacterial isolates. In particular, the procedure was tested with E. coli, K. pneumoniae, E. faecalis, and S. aureus (FIG. 3A). These bacteria cover both Gram-negative and Gram-positive species and represent clinically important multidrug-resistant pathogens that cause bloodstream infections and other bacterial infections. In the experiment, E. coli, E. faecalis, and K. pneumoniae were recovered with 50-60% efficiency as expected. However, recovery of S. aureus resulted in a lower recovery efficacy and a large batch-to-batch variation. The recovery rate was between 10% and 30%, compared to over 50% in other bacteria.

To explore the mechanism responsible for the lower recovery rate of S. aureus, the sedimentation step was repeated in isolated plasma (i.e., the majority of blood cells removed) and in buffer (i.e., no blood cells and blood proteins). In both conditions, the plasma and sediment portion had an approximately equal concentration of bacteria (FIG. 3B). Therefore, the reduction in recovery rate likely involved both blood cells and plasma proteins (e.g. clotting factors). S. aureus is uniquely known to agglutinate in blood and plasma through the action of bacterial clumping factors that interact with host proteins in blood.²⁰⁻²¹ Coagulated S. aureus may become passively entangled with red blood cell rouleaux in dextran, whereas this would not be an issue in RBC-depleted plasma (as tested in FIG. 3B).

To test the hypothesis that the reduced isolation efficiency is a result of S. aureus-mediated coagulation, an anticoagulant, argatroban, was provided into the mixture during the dextran sedimentation procedure. The results revealed that the recovery rate was restored to over 50% with 0.1 μM of argatroban (FIG. 3C). A higher argatroban concentration did not further improve the recovery, suggesting a small amount of anticoagulant is sufficient to eliminate the effect of the S. aureus-mediated coagulation. The experiment was also performed in E. coli to verify that anticoagulant treatment did not influence the dextran sedimentation efficiency in other bacteria. These results suggest the dextran sedimentation step is suitable for isolating common bacterial pathogens, and the addition of argatroban into the sedimentation tube enables processing of pathogens known to interact with blood cells and the coagulation cascade. The data in FIG. 3A-D represent mean±s.e.m (n=3). One-Way ANOVA with Tukey's HSD test. * P<0.05, NS=not significant.

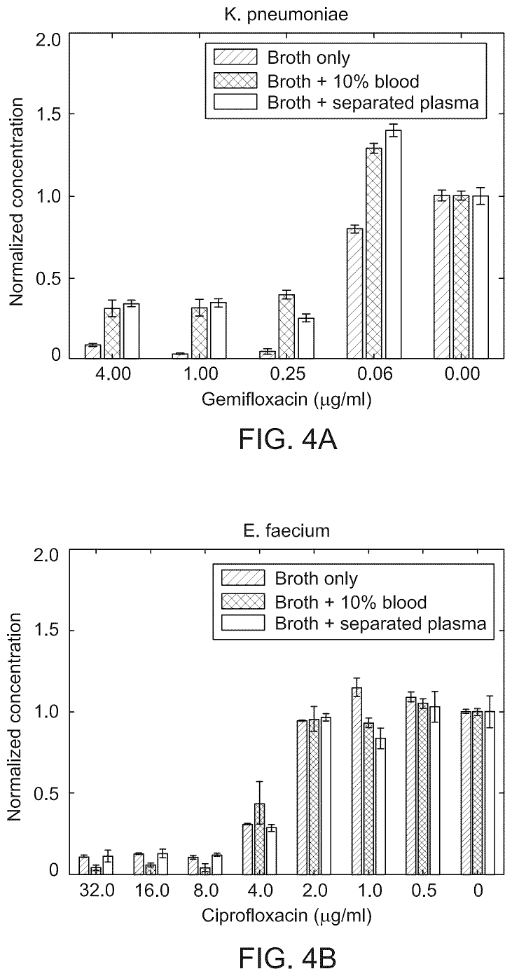

In embodiments, the disclosed techniques permit direct antimicrobial susceptibility testing without the time-limiting blood culture step. To evaluate if dextran and the remaining blood component influence the antimicrobial susceptibility testing result, antimicrobial susceptibility testing experiments were conducted with broth only, broth with 10% blood, and dextran-isolated plasma with MH broth at 1:1 ratio. The broth-only case represented a standard antimicrobial susceptibility testing condition. The broth with 10% blood was included to evaluate the influence of blood components (cells and proteins) on the minimum inhibitory concentration (MIC). The separated plasma mixed with MH broth at 1:1 ratio tested the effect of dextran and represented the antimicrobial susceptibility testing condition in the proposed workflow. The experiment was performed in K. pneumoniae (FIG. 4A) and E. faecium (FIG. 4B). The results indicate that the MIC was not affected by the inclusion of 10% blood or dextran separation protocol. For example, the E. faecium has a MIC between 4 and 8 μg/ml in all three conditions. These results further support direct antimicrobial susceptibility testing from whole blood samples using the disclosed workflows.

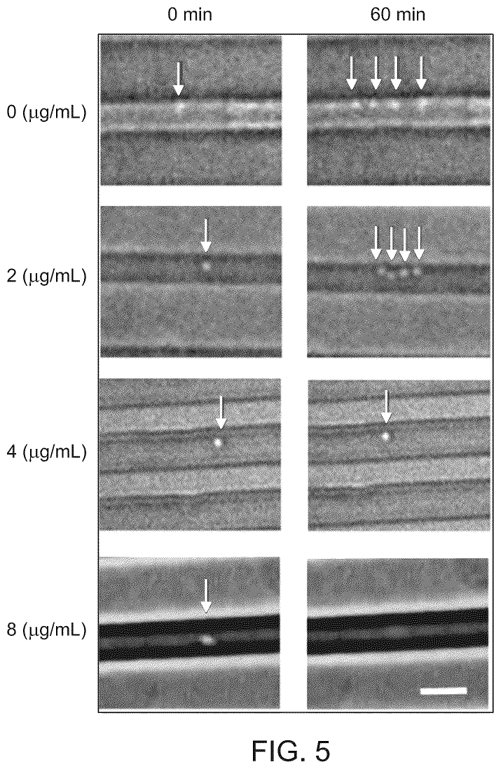

The disclosed workflows may be used for antimicrobial susceptibility testing using a microfluidic device. The microfluidic device trapped bacteria in one dimensional channels, and the bacteria were allowed to grow along the channel for phenotypic antimicrobial susceptibility testing. The antimicrobial susceptibility of an E. coli clinical isolate to ampicillin was tested as a demonstration (FIG. 5). The sample was separated into four tubes and mixed with different concentrations of antibiotics. The MIC of the bacteria strain was between 2 and 4 μg/ml. The growth of bacteria was only observed when the concentration of ampicillin was below 2 μg/ml. At a higher concentration (e.g., 8 μg/ml) of ampicillin, which is bacteriolytic, some bacteria were lysed during the duration of the experiment. The MIC value was below the susceptible breakpoint for Enterobacteriacea according to the Clinical and Laboratory Standards Institute (CLSI) guideline. The result was in categorical agreement with the clinical microbiology laboratory report. The data demonstrated the workflow for rapid diagnostics of bloodstream infections from whole blood to AST.

Separation of red blood cells (RBC) from whole blood can be performed prior to analysis or therapeutic use of less abundant cells, such as white blood cells or stem cells. While certain techniques use dextran sedimentation to separate cells present in blood, such as erythrocytes, the use of dextran in a bacterial sedimentation step to isolate bacteria in whole blood as disclosed herein is novel. Further, the use of an anticoagulant, e.g., argatroban, improves bacterial isolation for bacteria that interact with blood cells and that otherwise may be retained in a bottom layer 18 (see FIG. 0.1A). Thus, the disclosed techniques are more broadly applicable for isolation of pathogens of unknown types in a whole blood sample.

In the disclosed techniques, generally two layers are formed during sedimentation (e.g., gravimetric sedimentation), and removal and a simple one-step centrifugation of the top layer forms a centrifuged pellet retaining any bacteria in the original whole blood sample. The dextran in the sedimentation step may be provided in a 1:1 volume ratio with the whole blood sample, or in a range of about 0.8:1 (whole blood:dextran solution) to about 1:1.5 (whole blood:dextran solution) in embodiments. In one embodiment, the dextran solution contacted with the whole blood sample has a greater volume relative to a volume of the whole blood sample. The dextran may have a size of at least 75 kDa in an embodiment. In a particular embodiment, the dextran has a molecular weight of 100 kDa to 600 kDa or 200 kDa to 500 kDa. The dextran solution may be in a range of 1% to 10% (weight to volume) dextran.

While certain disclosed embodiments are discussed in the context of dextran sedimentation, in addition to or instead of dextran, other aggregating agents may be used in the sedimentation step. The other aggregating agents may be formulated using concentrations and may have molecular weight characteristics similar to those disclosed with respect to dextran. Examples of aggregating agents include, but are not limited to, high molecular weight polymeric molecules such as certain proteins like fibrinogen or gamma globulin; gelatin, and certain polysaccharides like dextran, hetastarch, pentastarch, and polyethylene glycol (PEG). The aggregating agent mixes and reacts with the biological sample to facilitate gravimetric sedimentation, which is functionally distinct from other polymeric additives known in the art, such as thixotropic gels and other solids, that facilitate differential sedimentation during centrifugation.

Further, certain disclosed embodiments are discussed in conjunction with an anticoagulant, such as argatroban. Additionally or alternatively, other thrombin inhibitors such as antithrombin, hirudin, dabigatran, lepirudin, desirudin, and bivalirudin may be used as part of a sedimentation solution, kit, or technique. Further, other anticoagulants, such as warfarin, heparin, acenocoumarol, phenprocoumon, atromentin, and phenindion may be used. In an embodiment, the anticoagulant is sodium polyanethole sulfonate (SPS). Thus, the anticoagulant may be a single anticoagulant or an anticoagulant mixture (e.g., SPS and argatroban) that is used in the presence of an aggregating agent (e.g., dextran) as provided herein.

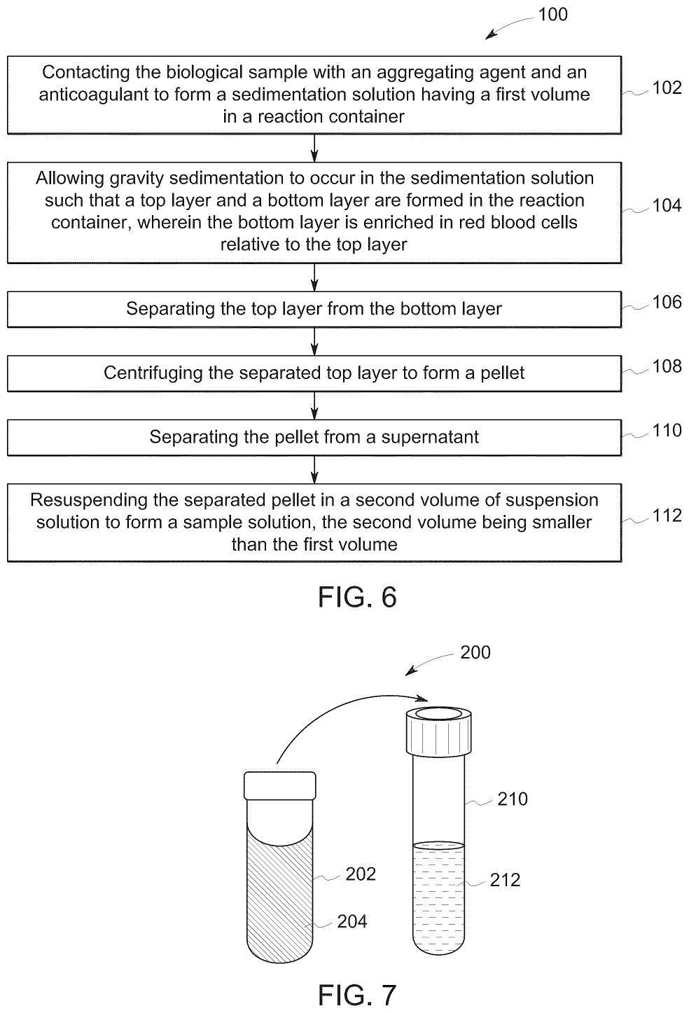

FIG. 6 is a flow diagram 100 of a method to isolate bacterial cells from a biological sample comprising red blood cells. At step 102, a biological sample is contacted with an aggregating agent and an anticoagulant to form a sedimentation solution. The biological sample maybe a blood sample, such as a whole blood sample, from a subject being tested for a bloodstream infection. The biological sample may be a sample of unknown infection status, and the infection status (e.g., presence or absence of bacteria) may be determined as disclosed herein. In an embodiment, the whole blood sample is provided with buffers, additives, or other additions to maintain the sample integrity. The aggregating agent and the anticoagulant may be added to the biological sample, e.g., mixed with the biological sample.

At step 104, gravimetric sedimentation of the sedimentation solution occurs such that a top layer and a bottom layer are formed in the sample processing container, wherein the bottom layer is enriched in red blood cells relative to the top layer. The red blood cells are sedimented into the bottom layer, while any pathogens present in the biological sample are retained in the top layer. The sedimentation may occur in 15 minutes or less or 30 minutes or less. In an embodiment, the sedimentation occurs in 60 minutes or less.

At step 106, the top layer is separated from the bottom layer. For example, the top layer can be moved into a separate container. The separated top layer is centrifuged at step 108 to form a pellet. The centrifugation may be a single step centrifugation. In an embodiment, the centrifugation is at speeds of 10,000 g or less and for 15 minutes or less. Supernatant is removed from the pellet at step 110, and the pellet is resuspended in a desired volume of suspicion solution at step 112. In an embodiment, the pellet may be washed before being prepared for downstream steps (e.g., resuspending, loading in an analysis device). The desired volume can be smaller than the starting volume of the biological sample and/or the volume of the sedimentation solution. Once resuspended, the sample solution can be provided to downstream analysis as disclosed herein. As provided herein, the centrifugation step is performed after gravimetric sedimentation of the biological sample, which is different from sedimentation steps caused by centrifugal forces, e.g., where sedimentation and centrifugation occur simultaneously.

FIG. 7 is an example of a kit 200 for sedimenting cells, e.g., to isolate bacteria present in a sample. A kit as referred to herein may include one or more reactants necessary for a given assay or test, set of directions to use the reactants present in the kit, any buffers necessary to maintain reaction conditions and other optional materials such as sample processing containers. The kit may include a container 202 that includes a reaction solution or mixture 204. In an embodiment, the reaction solution 204 includes an aggregating agent, such as dextran, in an appropriate concentration for mixing with a biological sample 212 that includes red blood cells. The reaction solution 204 may, in embodiments, include an anticoagulant premixed with the aggregating agent. However, in other embodiments, the kit 200 may include an aggregating agent and an anticoagulant in separate containers that are either mixed together and then added to the biological sample 212 or that are individually added to a sample processing container. In the illustrated embodiment, the reaction solution 204 is added to the container 210 that already holds the biological sample. However, in other embodiments, the sample processing container may be the container 202 holding one or both of the aggregating agent and the anticoagulant, or may be a separate, dedicated container of the kit 200.

The disclosed techniques include a workflow for single cell antimicrobial susceptibility testing at a clinically relevant concentration (10 cfu/mL). Sepsis diagnostics, however, could be as low as 1 cfu/mL. Notably, the isolated sample was separated into multiple tubes for testing various antibiotic conditions. The limit of detection of the workflow can be enhanced by further optimizing the workflow. For instance, the initial blood volume can be enhanced to increase the bacteria count in the sample. If necessary, a short pre-culture step (e.g., 2 hours) can be added in the workflow to increase the initial bacteria count. The efficiency of bacteria loading can also be enhanced by incorporating other microfluidic modules (e.g., electrokinetic trapping and enrichment). The disclosed techniques may be used for isolation of bacterial pathogens blood samples from patients with different clinical conditions, who have different cell distributions in their whole blood samples. For example, sepsis-induced effects may result in an elevated white blood cell count.

Technical effects of the invention include improved isolation and/or enrichment of bacteria from whole blood samples for pathogen identification and antimicrobial susceptibility testing. In particular, a dextran sedimentation step reduces the concentration of blood cells by four orders of magnitude in 20-30 minutes while maintaining the effective concentration of bacteria in the sample. Red blood cell depletion facilitates the downstream centrifugation-based enrichment step at a sepsis-relevant bacteria concentration. To avoid S. aureus-mediated coagulation, which reduces the overall recovery efficiency, a blood matrix effect disrupter or an anticoagulant, e.g., argatroban, can be incorporated into the mixture during the dextran sedimentation procedure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method to isolate bacterial cells from a biological sample comprising red blood cells, the method comprising:
contacting the biological sample with an aggregating agent and an anticoagulant to form a sedimentation solution having a first volume in a sample processing container;
allowing gravimetric sedimentation to occur such that a top layer and a bottom layer are formed in the sample processing container, wherein the bottom layer is enriched in red blood cells relative to the top layer;
isolating the top layer from the bottom layer;
centrifuging the isolated top layer to form a pellet;
separating the pellet from a supernatant; and
resuspending the separated pellet in a second volume of suspension solution to form a sample solution, the second volume being smaller than the first volume.

2. The method of claim 1, comprising loading the sample solution in a microfluidic analysis device.

3. The method of claim 1, comprising using the microfluidic analysis device to perform an antimicrobial susceptibility test.

4. The method of claim 1, comprising using the microfluidic analysis device to identify one or more pathogens in the sample solution.

5. The method of claim 1, wherein the aggregating agent comprises a high-molecular weight polymer.

6. The method of claim 5, wherein the aggregating agent is between 1-10% weight to volume in the solution.

7. The method of claim 1, wherein the contacting comprises contact the biological sample with the anticoagulant and the aggregating agent simultaneously.

8. The method of claim 1, wherein the anticoagulant comprises argatroban and the aggregating agent comprises dextran having a molecular weight of at least 100 kDa.

9. The method of claim 1, comprising washing the separated pellet prior to loading the sample solution into the microfluidic analysis device.

10. A kit to sediment cells in a biological sample, comprising:
an aggregating agent, the aggregating agent have a molecular weight of at least 100 kDa; and
an anticoagulant.

11. The kit of claim 10, wherein the aggregating agent is in solution.

12. The kit of claim 11, wherein the aggregating agent is between 1%-8% weight to volume in the solution.

13. The kit of claim 11, comprising the biological sample, wherein the biological sample is whole blood, and wherein a volume of the whole blood and a volume of the solution have a ratio in a range of 0.8:1 to 1:1.5.

14. The kit of claim 11, wherein the anticoagulant is in the solution.

15. The kit of claim 10, wherein the anticoagulant is a thrombin inhibitor.

16. The kit of claim 10, wherein the aggregating agent is hetastarch or gelatin.

17. The kit of claim 10, wherein the anticoagulant is argatroban.

18. The kit of claim 10, wherein the aggregating agent is dextran.

19. The kit of claim 10, comprising a sample processing container in which the aggregating agent and the anticoagulant are disposed.

20. A method to analyze whole blood, the method comprising:
forming a top layer and a bottom layer via gravimetric sedimentation in a sample processing container comprising a biological sample, an aggregating agent and an anticoagulant, wherein the bottom layer is enriched in red blood cells relative to the top layer;
centrifuging the top layer to form a pellet;
resuspending the separated pellet in a second volume of suspension solution to form a sample solution; and
analyzing the sample solution for a presence of bacteria.

21. The method of claim 20, wherein the sample solution is not cultured before the analyzing.
